(19) United States
(12) Patent Application Publication
(10) Pub. No.: US 2021/0000427 A1
(43) Pub. Date: Jan. 7, 2021

(54) Rendering Data Representative of Glycaemic Change Mediating Factors on Glycaemia

(71) Applicant: Quin Technology Ltd, London (GB)

(72) Inventors: Isabella Elisabeth Degen, London (GB); Cynthia Louise Williams, London (GB)

(21) Appl. No.: 16/969,271

(22) PCT Filed: Dec. 20, 2018
(86) PCT No.: PCT/GB2018/053708

§ 371 Date: Aug. 12, 2020

(30) Foreign Application Priority Data
Feb. 13, 2018 (GB) 1802325.9

(51) Int. Cl. A61B 5/00; A61B 5/145; A61B 5/11; G16H 20/60

(52) U.S. Cl. CPC A61B 5/7275; G16H 20/60; A61B 5/1118; A61B 5/14532

(57) ABSTRACT

A method and apparatus for rendering data representing the effect of glycaemic change mediating factors on glycaemia. The method comprises: recording a commencement time for a first glycaemic change mediating factor and an expected impact level for the first glycaemic change mediating factor, and plotting a first timeline from the recorded commencement time of the first glycaemic change mediating factor, representative of the estimated effect on glycaemia of the first glycaemic change mediating factor, based on the recorded expected impact level for the first glycaemic change mediating factor; wherein the rendering comprises: aligning the first plotted timeline to represent the estimated effect from a start point corresponding to a current time, and moving the first plotted timeline relative to the start point, as time progresses.

### BACKGROUND

### Technical Field

The present invention relates to methods of rendering data representing the effect of at least one glycaemic change mediating factor on glycaemia, for example the effect of a pharmacologically active agent on glycaemia or the effect of food on glycaemia. The present invention also relates to apparatus and computer program products in this field.

### Description of Related Art

Management of glycaemia is an ongoing challenge for people that suffer from diabetes. Taking of blood samples to regularly measure blood sugar levels gives people feedback on how well the balance between consumption of food, which tends to increase glycaemia, and the administration of pharmacologically active agents, for example insulin or synthetic insulin analogues which tend to reduce glycaemia, is controlling glycaemia to remain within a desired range. However, for many people, maintaining their blood sugar levels within a desired range is still a challenge as they go about their day to day life, given the complicated relationship between glycaemia and administration of pharmacologically active agents, food consumption and other factors such as activity level, the environment and each individual's endocrine system.

Part of the challenge is to recognise previous patterns representative of their personal responses as basis for an expected glycaemia, and to map recent administration of pharmacologically active agents, food consumption etc onto those patterns, to thereby make any estimations of the most suitable next action in terms of what to do with food or a pharmacologically active agent, and at what point in time.

Recording a history of glycaemia measurements and administration of pharmacologically active agents over time is known, as is apparatus for displaying such a recorded history. However, these known techniques and apparatus are not that useful in guiding decision making on the nature and timing of the most suitable next actions in management of glycaemia, since they do not reflect the expected impact of recent actions on glycaemia. Furthermore, since time variant factors are relevant to the future expected glycaemia, it is challenging to reflect these in a simple, and readily discernible manner, such as may be viewed on a portable device with limited screen size and resolution.

Example embodiments aim to address problems associated with existing related solutions, whether specifically mentioned above or which can otherwise be appreciated from the discussion herein.

### SUMMARY OF THE INVENTION

In one aspect there is provided a method of rendering data representing the effect of one or more glycaemic change mediating factors on glycaemia, the method comprising: recording a commencement time for a first glycaemic change mediating factor and an expected impact level for the first glycaemic change mediating factor, and plotting a first timeline from the recorded commencement time of the first glycaemic change mediating factor, representative of the estimated effect on glycaemia of the first glycaemic change mediating factor, based on the recorded expected impact level for the first glycaemic change mediating factor; wherein the rendering comprises: aligning the first plotted timeline to represent the estimated effect from a start point corresponding to a current time, and moving the first plotted timeline relative to the start point, as time progresses.

In one example, the first glycaemic change mediating factor is included in a group comprising: administration of a pharmacologically active agent, consumption of food, performance of physical activity, exposure to external environmental stimulus; and a hormonal factor.

In one example the first glycaemic change mediating factor comprises administration of a pharmacologically active agent, wherein the recorded commencement time comprises the time of administration of the pharmacologically active agent, and recording the expected impact level comprises recording dose information for the pharmacologically active agent; and wherein plotting the timeline of the estimated effect of the pharmacologically active agent on glycaemia takes place from the recorded start time for the administration of the pharmacologically active agent, and is based on the recorded dose information.

In one example the method further comprises recording a commencement time for a second glycaemic change mediating factor and an expected impact level for the second glycaemic change mediating factor, and plotting a second timeline from the recorded commencement time of the second glycaemic change mediating factor representative of the estimated effect on glycaemia of the second glycaemic change mediating factor, based on the recorded expected impact level for the second glycaemic change mediating factor.

In one example the rendering comprises: aligning the second plotted timeline to represent the estimated effect from a start point corresponding to a current time, and moving the second plotted timeline relative to the start point, as time progresses.

In one example the second glycaemic change mediating factor is included in a group comprising: administration of a pharmacologically active agent, consumption of food, performance of physical activity, exposure to external environmental stimulus; and a hormonal factor.

In one example the second glycaemic change mediating factor comprises consumption of food, and wherein the recorded commencement time comprises the time of consumption of the food, and recording the expected impact level comprises recording glycaemic load information for the food; and wherein plotting the timeline of the estimated effect of the consumption of the food on glycaemia takes place from the recorded start time for the consumption of the food, and is based on the recorded glycaemic load information.

In one example rendering the first and second timelines comprises aligning the timelines with one another. In one example the step of moving the plotted timelines together comprises rotating the plotted timelines about a rotation centre point.

In one example, the step of moving the plotted timelines together comprises rotating the plotted timelines about the rotation centre point, said rotation centre point being offset from the start point. In one example, the step of moving the plotted timelines together comprises rotating the plotted timelines about the rotation centre point at a predetermined rate over time.

In one example the step of aligning the plotted timelines comprises arranging one closer to a rotation centre point than the other, and rotating the plotted timelines around said rotation centre point.

In one example, the step of aligning the plotted timelines comprises scaling the plotted timelines so that each has the same relationship between time and rotational angle.

In one example the step of aligning the plotted timelines comprises scaling the plotted timelines so that each is rendered around a circle in which one complete revolution corresponds to a predetermined time period.

In one example, the predetermined time period is one day, or less than one day, for example less than 18 hours, less than 12 hours, or less than 6 hours. In one example, the predetermined time period is 1 hour, or more than one hour, for example more than 2 hours, more than 3 hours, more than 4 hours or more than 6 hours. In one example the predetermined time period is between 4 and 24 hours, for example 6 or 12 hours.

In one example the method further comprises removing a previous estimated effect from the rendering, as time progresses and the plotted timeline moves past the start point.

In one example the rendering comprises providing an indicator on a plotted timeline, indicating the estimated effect on glycaemia, and corresponding to the current direction and strength of the estimated effect on glycaemia.

In one example, the indicator is selected from a first group comprising: a first upward indicator corresponding to a relatively strong raising effect, and a second upward indicator corresponding to a relatively weak raising effect and a third upward indicator corresponding to a neutral effect. In one example the indicator is selected from a second group comprising a first downward indicator corresponding to a relatively strong lowering effect, and a second downward indicator corresponding to a relatively weak lowering raising effect and a third downward indicator corresponding to a neutral effect.

In one example, the step of plotting a timeline of the estimated effect on glycaemia comprises determining the length of time over which the effect is estimated to exist, and scaling the length of the timeline. In one example the step of plotting a timeline of the estimated effect on glycaemia comprises determining the length of time over which the effect is estimated to exist, and scaling the length of the correspondingly to end a the point at which the effect is estimated to no longer exist.

In one example the step of plotting a timeline of the estimated effect on glycaemia from the recorded commencement time comprises indicating the strength of the effect along the timeline by gradation of the effect along the timeline.

In one example the gradation varies from indicating a strong effect to a weak effect along the timeline. In one example the gradation comprises shading, such as strong to weak shading along the timeline to correspond to the strength of the effect.

In one example the method comprises recording a commencement time for one or more further glycaemic change mediating factors and an expected impact level for each of said the further glycaemic change mediating factors, and plotting one or more further timelines from the recorded commencement time of the further glycaemic change mediating factor or factors, representative of the estimated effect on glycaemia of the further glycaemic change mediating factor, based on the recorded expected impact level for the further glycaemic change mediating factor or factors; wherein the rendering comprises: aligning the one or more further plotted timelines to represent the estimated effect from a start point corresponding to a current time, and moving the one or more further plotted timelines relative to the start point, as time progresses.

In one example the method comprises a further instance of recording a commencement time for one or more further glycaemic change mediating factors and an expected impact level for each of said the further glycaemic change mediating factors, and plotting one or more further timelines from the recorded commencement time of the further glycaemic change mediating factor or factors, representative of the estimated effect on glycaemia of the further glycaemic change mediating factor, based on the recorded expected impact level for the further glycaemic change mediating factor or factors; and wherein the rendering comprises: aligning all the plotted timelines to show the estimated effects from a start point representative of the current time, and moving all the plotted timelines together, relative to the start point, as time progresses.

In one example the aligning comprises arranging one or more of said timelines closer to the centre of rotation than the others, such that the plotted timelines run alongside one another.

In one example, the aligning comprises scaling one of the plotted timelines in a non-time dimension, such that the timeline corresponding to the stronger effect is more prominent than the timeline corresponding to the weaker effect, when compared at the start point. In one example, the scaling comprises reducing the timeline corresponding to the weaker effect such that said timeline is narrower than that corresponding to the weaker effect. In one example the aligning comprises scaling both plotted timelines in a non-time dimension so as to represent them in a width corresponding that of a previously-plotted timeline.

In one example, the aligning comprises scaling one of the plotted timelines in a non-time dimension, such that the timeline corresponding to the stronger effect is more prominent than the timeline corresponding to the weaker effect, when compared at the start point. In one example, the scaling comprises reducing the timeline corresponding to the weaker effect such that said timeline is narrower than that corresponding to the weaker effect. In one example the aligning comprises scaling both plotted timelines in a non-time dimension so as to represent them in a width corresponding that of a previously-plotted timeline.

In one example the plotted timelines are distinguishable such that the direction of the effect on glycaemia is discernible. In one example the plotted timelines are colour coded such that the direction of the effect on glycaemia is discernible. In one example, a plotted timeline corresponding to a raising effect on glycaemia is provided in a first colour, and a plotted timeline corresponding to a lowering effect on glycaemia is provided in a second colour, distinguishable from the first colour.

In another aspect there is provided an apparatus for displaying data representing the effect of one or more glycaemic change mediating factors on glycaemia, the apparatus comprising: a display; a data input unit arranged receive and record a commencement time for a first glycaemic change mediating factor and an expected impact level for the first glycaemic change mediating factor; a data processing unit arranged to plot a first timeline from the recorded commencement time of the first glycaemic change mediating factor, representative of the estimated effect on glycaemia of the first glycaemic change mediating factor, based on the recorded expected impact level for the first glycaemic change mediating factor; and an output unit arranged to render on the display, an aligned first timeline showing the estimated effects from a start point corresponding to a current time, and to move the first timeline, relative to the start point on the display, as time progresses.

In one example the data input unit is arranged to further record a commencement time for a second glycaemic change mediating factor and an expected impact level for the second glycaemic change mediating factor; the data processing unit is arranged to plot a second timeline from the recorded commencement time of the second glycaemic change mediating factor representative of the estimated effect on glycaemia of the second glycaemic change mediating factor, based on the recorded expected impact level for the second glycaemic change mediating factor; and the output unit is arranged to render on the display an aligned second timeline to represent the estimated effect from a start point corresponding to a current time, and to move the second timeline, relative to the start point, as time progresses.

In one example the output unit is arranged to provide, to the display, aligned plotted timelines generated according to the method as described herein, for example according to the method of any of the aspects or examples above.

In one example the apparatus comprises a portable electronic device. In one example the portable electronic device comprises a portable computing device, suitably a smartphone.

In one example the output unit is arranged to provide, to the display, a graphical representation of the aligned plotted timelines that is scaled to be readable in a widget area that comprises a portion of the display.

In one example the display comprises an area less than 16 cm by 8 cm. In one example the widget area comprises less than half of the display, for example less than quarter of the display. For example the widget area comprises an area on the display of less than 6 cm by 6 cm, for example 4 cm by 4 cm. For example the widget area comprises an area on the display of less than 500×500 pixels.

In another aspect there is provided a tangible non-transient computer-readable storage medium having recorded thereon instructions which, when implemented by a computer device, cause the computer device to carry out steps according to a method as described herein, for example according to the method of any of the aspects or examples above.

In one example there is provided a tangible non-transient computer-readable storage medium having recorded thereon instructions which, when implemented by a portable computing device, cause the portable computing device to carry out steps according to a method as described herein, for example according to the method of any of the aspects or examples above.

According to other aspects there is provided a method, an apparatus and a computer program product as set forth in the appended claims. Other features of the invention will be apparent from the dependent claims, and the description which follows.

### BRIEF DESCRIPTION OF THE DRAWINGS

For a better understanding of the invention, and to show how example embodiments may be carried into effect, reference will now be made to the accompanying drawings in which:

FIG. 1 is a schematic view of an example network including an example computer device;

FIG. 2 is a schematic view of the example network including the example computer device in more detail;

FIG. 3 is an example screen shot illustrating an output of a method of rendering data representing the effect of a glycaemic change mediating factors on glycaemia;

FIG. 4 is an example screen shot illustrating another output of a method of rendering data representing the effect of a glycaemic change mediating factors on glycaemia; and

FIG. 5 is flowchart illustrating an example method of operating the example computer device in order to render data representing the effect of a glycaemic change mediating factors on glycaemia.

### DETAILED DESCRIPTION OF THE EXAMPLE EMBODIMENTS

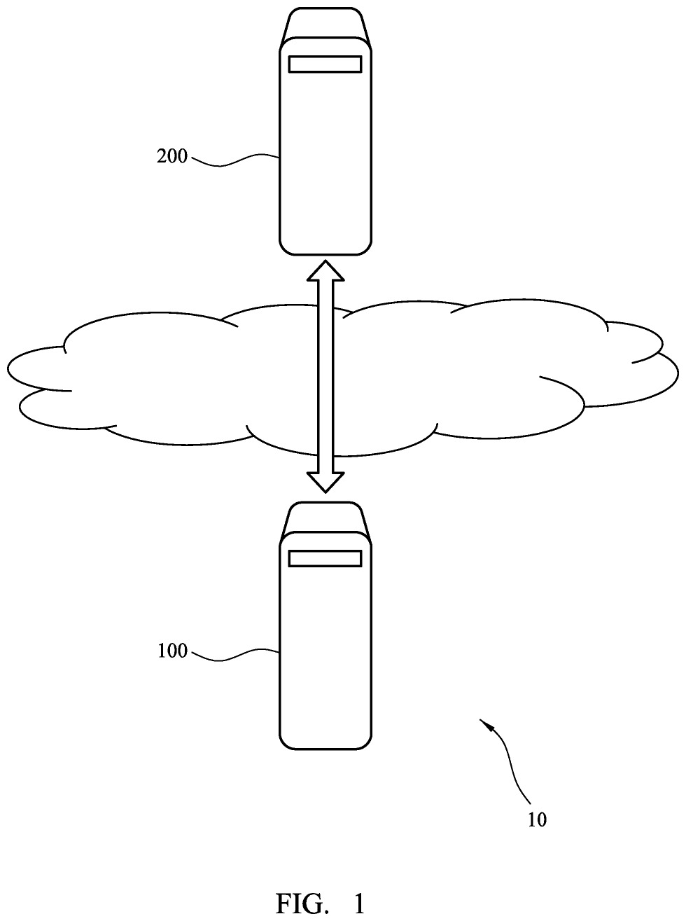

FIG. 1 is a schematic view of an example network 10 including an example computer device 200. The network 10 also includes another computer device 100. Using client-server terminology, the another computer device 100 is a client device, while the computer device 200 is a server device. For simplicity and without limitation, the computer device 200 is referred to herein as the server device 200 and the another computer device 100 is referred to herein as the client device 100. The client device 100 and the server device 200 are arranged to communicate using the network 10. The network 10 may be a private network, a virtual private network, an intranet, a cloud, or the Internet.

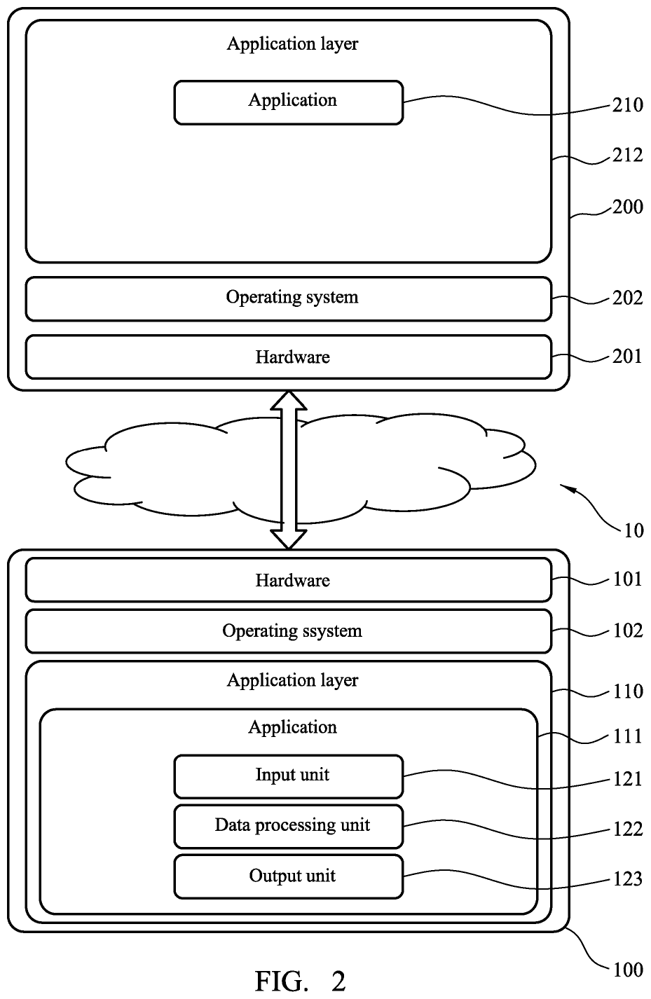

FIG. 2 is a schematic view of the example network 10 including the example computer device 200, in more detail.

The client device 100 may take any suitable form factor. As examples, the client device 100 may be a desktop computer, a portable computing device, laptop, tablet, smartphone, wearable device, or an emulated virtual device on any appropriate host hardware. The client device 100 comprises a layer of hardware (H/W) 101, which suitably includes memory, processors (CPU central processor units), I/O input/output interfaces (e.g. NIC network interface cards, USB universal serial bus interfaces, etc.), storage (e.g. solid state non-volatile storage or hard disk drive), and so on. An operating system 102, for example iOS 10, runs on the hardware layer 101 to provide a runtime environment for execution of user processes and applications. This runtime environment typically provides resources such as installed software, system services, drivers and files, in particular allowing application programs to be executed in the application layer 110.

The server device 200 may also take any suitable form factor, as described above with respect to the client device 100. As examples, the server device 200 may be a desktop computer, a portable computing device, laptop, tablet, smartphone, wearable device, or an emulated virtual device on any appropriate host hardware. The server device 200 comprises a layer of hardware (H/W) 201, which suitably includes memory, processors (CPU central processor units), I/O input/output interfaces (e.g. NIC network interface cards, USB universal serial bus interfaces, etc.), storage (e.g. solid state non-volatile storage or hard disk drive), and so on. An operating system 202, for example Windows 10, runs on the hardware layer 201 to provide a runtime environment for execution of user processes and applications. This runtime environment typically provides resources such as installed software, system services, drivers, and files, in particular allowing application programs to be executed in the application layer 210.

The client device 100 comprises an application program 120 that enables a user to collect information relevant to glycaemia, and which is configured to undertake operations thereafter which enable the client device 100 to provide a visual indication to the user which may be helpful for monitoring and prediction of likely changes in glycaemia, in particular by rendering data representative of the effect of one or more glycaemic change mediating factors. The client device 100 comprises an I/O device in the form of a touch screen display as part of the hardware 101 in order to facilitate such user interactions.

Within the application program 120 is a functional unit arranged to collect user data, referred to as an input unit 121. The input unit receives and records commencement times for first, second and subsequent glycaemic change mediating factors, together which an expected impact level for each of the inputted glycaemic change mediating factors.

The input unit 121 records the time, which for example may be a current time, time in the past, or expected future time as the commencement time for a first glycaemic change mediating factor. For example, if the first glycaemic change mediating factor comprises administration of insulin mediation, the input unit records the commencement time as the time at which the mediation is taken by the user stores this along with type and dosage information such as may be input by the user.

A data processing unit 122 within the application program 120 is configured to plot a first timeline, from the recorded commencement time of the first glycaemic change mediating factor, representative of the estimated effect on glycaemia of the first glycaemic change mediating factor, based on the recorded expected impact level for the first glycaemic change mediating factor. The expected impact level on glycaemia is determined based on the input type and dosage information, in combination with clinically determined efficacy information, which may be based on population-based statistical models alone, or modified based on information about the user's expected response, as available to the application program 120 either locally or with reference to the server device 200.

The application program 120 comprises an output unit 123, arranged to render on the touch screen display of the client device 100, an aligned first timeline showing the estimated effects from a start point corresponding to a current time, and to move the first timeline, relative to the start point on the display, as time progresses. In the example of administration of insulin medication as above, the client device plots a first timeline starting from the recorded commencement time of the first glycaemic change mediating factor according to when the medication is taken, and representative of the estimated effect on glycaemia of the medication over time. For rendering on the client device, the plotted timeline is aligned to represent the estimated effect from a start point corresponding to a current time. Thereafter, as time progresses the plotted timeline is moved relative to the start point.

As can be appreciated, insulin medication may have a significant estimated effect on glycaemia, but other factors that occur sporadically, and on a periodic basis will also be relevant. The example embodiments of the client device are therefore are adapted, according to operation of the application program 110 to record a commencement time for a second glycaemic change mediating factor and an expected impact level for the second glycaemic change mediating factor, in a similar manner as described for the first glycaemic change mitigating factor.

Based on this, it is possible to plot a second timeline from the recorded commencement time of the second glycaemic change mediating factor representative of the estimated effect on glycaemia of the second glycaemic change mediating factor in a similar manner to the description above. In this case the first and second timelines are aligned with one another and move together relative to the start point, as time progresses. In this way the potentially different effects on glycaemia may be easily recognised over time, simplifying a decision making process on whether to take further specific glycaemia control actions.

In the above example where the first glycaemic change mediating factor comprises administration of a pharmacologically active agent in the form of an insulin medication, the effect is generally to reduce glycaemia, whereas consumption of food as the second glycaemic change mediator has the effect of generally increasing glycaemia, so the extent to which the effects may balance out, and over what timeframe each may be relevant can be readily understood.

It will be understood that models exist for the impact of various medicaments. In one example the second glycaemic change mediating factor comprises consumption of food, and wherein the recorded commencement time comprises the time of consumption of the food, and recording the expected impact level comprises recording glycaemic load information for the food; and wherein plotting the timeline of the estimated effect of the consumption of the food on glycaemia takes place from the recorded start time for the consumption of the food, and is based on the recorded glycaemic load information.

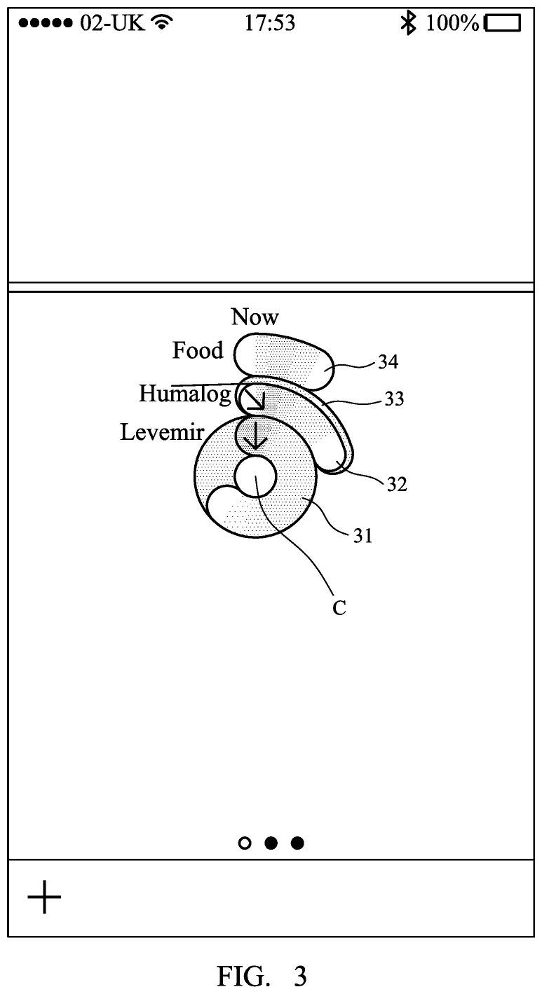

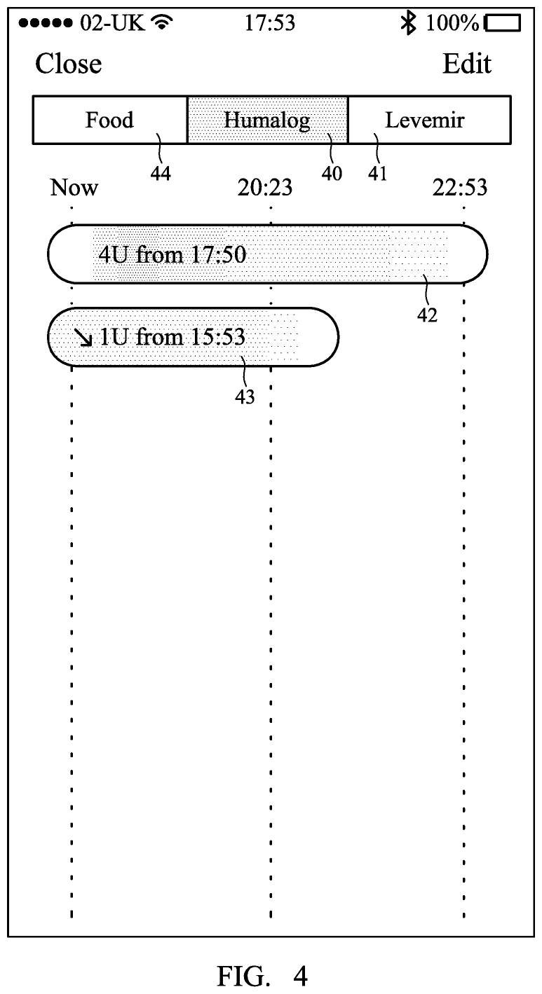

FIGS. 3 and 4 show example screenshots illustrating an output of a method of rendering data representing the effect of a glycaemic change mediating factors on glycaemia on a client device 100 in the form of a smartphone. The example screen shots are particularly advantageous when viewed on a small display area, for example when aligned plotted timelines are scaled to be readable in a widget area that comprises a portion of a hand-held display.

In the screenshot shown in FIG. 3, first, second, third and fourth timelines 31, 32, 33, 34 are provided as arcs of a circle, aligned with one another and the current time “Now” at the top centre. The first through fourth timelines 31, 32, 33, 34 are to be moved by rotation. Moving the plotted timelines together comprises rotating the plotted timelines about a rotation axis at the centre point C, in this example in the anti-clockwise direction and takes place at a predetermined rate over time. In order to make the plotted timelines readily understandable, aligning the plotted timelines is performed in a way that includes scaling the plotted timelines so that each has the same relationship between time and rotational angle, so that each is rendered around a circle in which one complete revolution corresponds to a predetermined time period. The scaling factor may be determined, for example according to user preference, such that the predetermined time period is one day, for example 12 hours which will correspond to a time frame relevant to the remainder of most people's daily activities, or as shown in FIG. 3, 6 hours as a time frame relevant to the next major portion of the day, or maximum time between meals.

Since previous estimated effects cannot be changed by future actions, they are not relevant to current decisions on glycaemia management, and are therefore removed from the timelines as time progresses and the plotted timelines are moved past the start point.

FIG. 3 illustrates an embodiment in which the rendering comprises providing an indicator on a plotted timeline, indicating the estimated effect on glycaemia. The indicator corresponds to the current direction and strength of the estimated effect on glycaemia. The fourth plotted timeline 34, labelled “Food”, is provided in a particular colour, in this case green, to indicate a generally rising effect. The strength of the effect over time decreases and this is indicated by the fade from dark to light. The first and second timelines 31, 32 labelled “Levemir” and “Humalog” correspond to administration of medicaments with these names, and are provided in a particular colour, in this case blue, to indicate a generally lowering effect. The colours used to indicate generally raising and lowering effects are chosen to be contrasting. The strength of these effects over time also decreases and this is correspondingly indicated by the fade from dark to light.

In addition, when particularly pronounced effect is expected at a particular short time interval a further indicator is provided, selected from a first group comprising: a first upward indicator corresponding to a relatively strong raising effect, and a second upward indicator corresponding to a relatively weak raising effect and a third upward indicator corresponding to a neutral effect. Corresponding downward indicators are also used where appropriate. In FIG. 3 the second timeline 32 labelled “Humalog” is provided with an indicator for a relatively weak lowering effect at the current time “Now”, whereas the first timeline labelled “Levemir” 31 is provided with an indicator for a relatively strong lowering effect at the current time “Now”.

The third timeline 33 is also associated with an administration of Humalog medicament, but based on the time/dosage information the effect of this administration is minor in comparison to the effect of the administration corresponding to the second timeline 32. For this reason the second and third timelines 32, 33 are shown alongside one another, with the major effect produced by the different instances of the same glycaemic change mediating factor shown at increased prominence. Here the prominence of the second timeline 32 is increased by its relatively larger width compared to the third timeline 33.

FIG. 4 shows linear timelines 41, 42 with a time scaling, colouration, a fade effect. These effects are identified as being caused by “Humalog”, as per the highlighted menu item in the centre of the display. Similar to FIG. 3, two administrations of Humalog are rendered in two plotted timelines, with in this case the more significant effect shown as the uppermost between the two timelines. In this example the timelines 41, 42 more to the left as time progresses. Menu items 41, 44 are also shown to enable “Levemir” and “Food” timelines to be selected for rendering on the display, alongside the highlighted menu item 40 indicating that the current display features timelines relating to the effect of Humalog.

As will be appreciated, the screenshots show the entirety of a smartphone display, but the characteristics of the rendered timelines makes them particularly suited for display on an even smaller area such as that of a widget or notification panel.

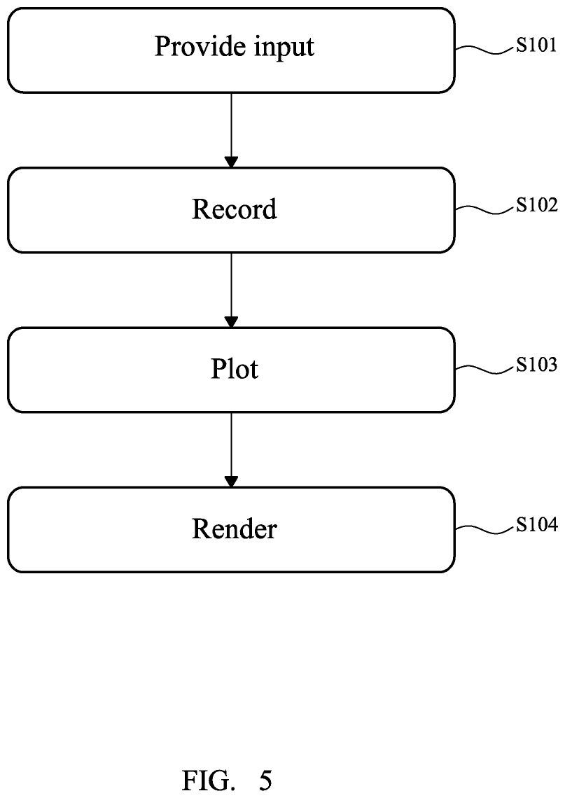

FIG. 5 is a flowchart illustrating an example method of operating an example computer device in order to render data representing the effect of one or more glycaemic change mediating factors on glycaemia.

At S101, a user of the device provides an input that describes a glycaemic change mediating factor that is relevant to them. For example, the user may have self administered a pharmacologically active agent such as taking rapid acting insulin. The user may input the time of taking the medication, the type of medication and the dosage amount.

At step 102, the device records a commencement time for the glycaemic change mediating factor and an expected impact level for the first glycaemic change mediating factor. At step 103, based on the recorded information the device plots a first timeline from the recorded commencement time of the first glycaemic change mediating factor, the timeline representative of the estimated effect on glycaemia of the first glycaemic change mediating factor.

At step S104, the device renders the plotted timeline by aligning the first plotted timeline to represent the estimated effect from a start point corresponding to a current time, and moving the first plotted timeline relative to the start point, as time progresses.

A wide range of factors are understood to act as glycaemic change mediating factors. The main factors are administration of pharmacologically active agents such as insulin medicaments, consumption of food, performance of physical activity, exposure to external environmental stimulus, and hormonal factors. Of particular relevance among hormonal factors are the endocrine system hormones, such as cortisol, glucagon and adrenaline. Such hormones are relevant to regulation of glycaemia and furthermore their levels in the body can be linked to naturally occurring variation over time, or to response to external stimuli such as stress. The method may further comprise going through the abovementioned steps for a second glycaemic change mediating factor, such that at the end of the process a second plotted timeline, related to the second glycaemic change mitigating factor is rendered, such that the second plotted timeline is aligned with the first. In this way the second plotted timeline can represent the estimated effect on glycaemia of the second factor from a start point corresponding to a current time. Thus the first and second rendered timelines move together relative to the start point, as time progresses. The method may be performed each time a significant glycaemic change mediating factor is encountered, so that at all time the user may be able to understand from the rendered timeline(s) whether previous actions linked to those factors will be operating to increase or decrease glycaemia. Furthermore, how long those effects can be expected to last, and their relative strength may be readily determined by inspection of the rendered timeline(s).

In the example method above, the steps may be repeated for a second glycaemic change mediating factor that comprises consumption of food. In this instance, the recorded commencement time comprises the time of consumption of the food, and the recorded the expected impact level comprises glycaemic load information for the food. Plotting the timeline of the estimated effect of the consumption of the food on glycaemia takes place from the recorded start time for the consumption of the food, and is based on the recorded glycaemic load information. In one example embodiment the device may store relevant information about the food, and its typical expected effect on glycaemia local, or may access a remote server to request such information. In some embodiments, personal information such as age, weight and experimentally determined insulin response of the user to one or more of the factors, or experimentally determined insulin response of the general population may also be factored in to the plotted timeline.

At least some elements discussed herein may be constructed, partially or wholly, using dedicated special-purpose hardware. Terms such as ‘component’, ‘module’ or ‘unit’ used herein may include, but are not limited to, a hardware device, such as a Field Programmable Gate Array (FPGA) or Application Specific Integrated Circuit (ASIC), which performs certain tasks.

At least some elements may be may be configured to reside on an addressable storage medium and be configured to execute on one or more processors. That is, the elements may be implemented in the form of a tangible computer-readable storage medium having recorded thereon instructions that are, in use, executed by a computer or other suitable device. The elements may include, by way of example, components such as software components, object-oriented software components, class components and task components, processes, functions, attributes, procedures, subroutines, segments of program code, drivers, firmware, microcode, databases, data structures, tables, arrays, and variables. The tangible medium may take any suitable form, but examples include solid-state memory devices (ROM, RAM, EPROM, EEPROM, etc.), optical discs (e.g. Compact Discs, DVDs, and others), magnetic discs, magnetic tapes and magneto-optic storage devices.

The example embodiments have been described with reference to the example components, modules and units discussed herein. Where appropriate, these functional elements may be combined into fewer elements or separated into additional elements. In some cases, the elements are distributed over a plurality of separate computing devices that are coupled by a suitable communications network, including any suitable wired networks or wireless networks. Throughout this specification, the term “comprising” or “comprises” may mean including the component(s) specified but is not intended to exclude the presence of other components.

Although a few preferred embodiments have been shown and described, it will be appreciated by those skilled in the art that various changes and modifications might be made without departing from the scope of the invention, as defined in the appended claims.

1-19. (canceled)

20. A method of rendering data representing the effect of one or more glycaemic change mediating factors on glycaemia, the method comprising:
recording a commencement time for a first glycaemic change mediating factor and an expected impact level for the first glycaemic change mediating factor; and
plotting a first timeline from the recorded commencement time of the first glycaemic change mediating factor, representative of the estimated effect on glycaemia of the first glycaemic change mediating factor, based on the recorded expected impact level for the first glycaemic change mediating factor;
wherein the rendering comprises:
aligning the first plotted timeline to represent the estimated effect from a start point corresponding to a current time; and
moving the first plotted timeline relative to the start point, as time progresses.

21. The method of claim 20, wherein the first glycaemic change mediating factor is included in a group comprising:
administration of a pharmacologically active agent;
consumption of food;
performance of physical activity;
exposure to external environmental stimulus; and
a hormonal factor.

22. The method of claim 20, wherein:
the first glycaemic change mediating factor comprises administration of a pharmacologically active agent;
the recorded commencement time comprises the time of administration of the pharmacologically active agent;
the recording the expected impact level comprises recording dose information for the pharmacologically active agent; and
the plotting the timeline of the estimated effect of the pharmacologically active agent on glycaemia takes place from the recorded start time for the administration of the pharmacologically active agent, and is based on the recorded dose information.

23. The method of claim 20, further comprising:
recording a commencement time for a second glycaemic change mediating factor and an expected impact level for the second glycaemic change mediating factor; and
plotting a second timeline from the recorded commencement time of the second glycaemic change mediating factor representative of the estimated effect on glycaemia of the second glycaemic change mediating factor, based on the recorded expected impact level for the second glycaemic change mediating factor;
wherein the rendering comprises:
aligning the second plotted timeline to represent the estimated effect from a start point corresponding to a current time; and
moving the second plotted timeline relative to the start point, as time progresses.

24. The method of claim 23, wherein the second glycaemic change mediating factor is included in a group comprising:
administration of a pharmacologically active agent;
consumption of food;
performance of physical activity;
exposure to external environmental stimulus; and
a hormonal factor.

25. The method of claim 24, wherein:
the second glycaemic change mediating factor comprises consumption of food;
the recorded commencement time comprises the time of consumption of the food;
the recording the expected impact level for the second glycaemic change mediating factor comprises recording glycaemic load information for the food; and
the plotting the second timeline of the estimated effect of the consumption of the food on glycaemia takes place from the recorded start time for the consumption of the food, and is based on the recorded glycaemic load information.

26. The method of claim 25, wherein the rendering the first and second timelines comprises aligning the plotted timelines with one another.

27. The method of claim 26, wherein the aligning the plotted timelines together comprises rotating the plotted timelines about a rotation center point.

28. The method of claim 26, wherein the aligning the plotted timelines comprises:
arranging one of the plotted timelines closer to a rotation center point than the other; and
rotating the plotted timelines around said rotation center point.

29. The method of claim 26, wherein the aligning the plotted timelines comprises scaling the plotted timelines so that each of the plotted timelines is rendered around a circle in which one complete revolution corresponds to a predetermined time period.

30. The method of claim 20, further comprising removing a previous estimated effect from the rendering, as time progresses and the plotted timeline moves past the start point.

31. The method of claim 20, wherein the rendering comprises providing an indicator on a plotted timeline, indicating the estimated effect on glycaemia, and corresponding to the current direction and strength of the estimated effect on glycaemia.

32. The method of claim 20, wherein the plotting the first timeline of the estimated effect on glycaemia from the recorded commencement time comprises indicating the strength of the effect along the first timeline by gradation of the effect along the first timeline.

33. The method of claim 20, further comprising:
recording a commencement time for one or more further glycaemic change mediating factors and an expected impact level for each of said the further glycaemic change mediating factors; and
plotting one or more further timelines from the recorded commencement time of the further glycaemic change mediating factor or factors, representative of the estimated effect on glycaemia of the further glycaemic change mediating factor, based on the recorded expected impact level for the further glycaemic change mediating factor or factors;
wherein the rendering comprises:
aligning the one or more further plotted timelines to represent the estimated effect from a start point corresponding to a current time; and
moving the one or more further plotted timelines relative to the start point, as time progresses.

34. An apparatus for outputting data representing the effect of one or more glycaemic change mediating factors on glycaemia, the apparatus comprising:
a display;
a data input circuit arranged to receive and record a commencement time for a first glycaemic change mediating factor and an expected impact level for the first glycaemic change mediating factor;
a data processing circuit arranged to plot a first timeline from the recorded commencement time of the first glycaemic change mediating factor, representative of the estimated effect on glycaemia of the first glycaemic change mediating factor, based on the recorded expected impact level for the first glycaemic change mediating factor; and
an output circuit arranged to:
render on the display, an aligned first timeline showing the estimated effects from a start point corresponding to a current time; and
move the first timeline, relative to the start point on the display, as time progresses.

35. The apparatus of claim 34, wherein:
the data input circuit is further arranged to record a commencement time for a second glycaemic change mediating factor and an expected impact level for the second glycaemic change mediating factor;
the data processing circuit is further arranged to plot a second timeline from the recorded commencement time of the second glycaemic change mediating factor representative of the estimated effect on glycaemia of the second glycaemic change mediating factor, based on the recorded expected impact level for the second glycaemic change mediating factor; and
the output circuit is further arranged to:
render on the display an aligned second timeline to represent the estimated effect from a start point corresponding to a current time; and
move the second timeline, relative to the start point, as time progresses.

36. The apparatus of claim 34, wherein the output circuit is arranged to provide, to the display, aligned plotted timelines generated by rotating the plotted timelines about a rotation center point.

37. The apparatus of claim 36, wherein the apparatus comprises a portable electronic device.

38. The apparatus of claim 34, wherein the output circuit is further arranged to provide, to the display, a graphical representation of the aligned plotted timelines that is scaled to be readable in a widget area that comprises a portion of the display.

39. A non-transitory computer-readable storage medium storing software instructions which, when implemented by a computer device, causes the computer device to:
record a commencement time for a first glycaemic change mediating factor and an expected impact level for the first glycaemic change mediating factor; and
plot a first timeline from the recorded commencement time of the first glycaemic change mediating factor, representative of the estimated effect on glycaemia of the first glycaemic change mediating factor, based on the recorded expected impact level for the first glycaemic change mediating factor;
wherein when implemented by the computer device, the software instructions implement the rendering by:
aligning the first plotted timeline to represent the estimated effect from a start point corresponding to a current time; and
moving the first plotted timeline relative to the start point, as time progresses.
